(19) United States
(12) Patent Application Publication
(10) Pub. No.: US 2011/0225100 A1
(43) Pub. Date: Sep. 15, 2011

(54) SYSTEM, METHOD AND INTERFACE DISPLAY FOR VERIFYING AND MANAGING DISTRIBUTION AND SALES OF MEDICINE

(75) Inventors: Puneet M. Sangal, Lebanon, NH (US); Nathan J. Sigworth, Hamden, CT (US); N. Taylor Thompson, Greensboro, NC (US); Sarah M. Hine, Southbury, CT (US)

(73) Assignee: PHARMASECURE, INC., Lebanon, NH (US)

(21) Appl. No.: 12/973,879

(22) Filed: Dec. 20, 2010

Related U.S. Application Data
(60) Provisional application No. 61/313,265, filed on Mar. 12, 2010.

(51) Int. Cl. G06Q 10/00; B65D 85/00; G06F 3/048

(52) U.S. Cl. 705/318; 206/459.5; 715/771

(57) ABSTRACT

A system, method and graphical user interface display for validating, monitoring and otherwise reviewing the sales and distribution of medicines, to reduce the instances of counterfeit medicines. A pharmaceutical company provides medicines to users, either directly or through representatives for the pharmaceutical company. The products have associated identifying codes that are used to track the sales of the products. The system provides verifications of the medicines, as well as statistical analysis of the sales and distribution. The verifications and comparisons of the distribution and sales of the medicines can be reviewed by the user or on a dashboard user interface. The system further allows pharmaceutical companies provided the medicines to provide response messages to users.

### RELATED APPLICATIONS

This application claims the benefit of U.S. Provisional Patent Application Ser. No. 61/313,265, filed Mar. 12, 2010, entitled SYSTEM, METHOD AND INTERFACE DISPLAY FOR VERIFYING AND MANAGING DISTRIBUTION AND SALES OF MEDICINE, the entire disclosure of which is herein incorporated by reference.

### FIELD OF THE INVENTION

This invention relates to systems and methods for managing medicines and other medical drugs provided by a pharmaceutical company. More particularly, it relates to monitoring and managing the distribution and sales of the medicines.

### BACKGROUND OF THE INVENTION

There is a major problem related to the distribution, sales and use of counterfeit drugs and other pharmaceutical products. In many countries around the world, medicines that are distributed and sold are fake, i.e. counterfeit. These medicines are deliberately and fraudulently mislabeled with respect to identity and/or source. They can range from unknown random combinations of toxic substances to ineffective, inactive preparations, such as lacking an active ingredient. The results of counterfeit drugs are that either there is an ineffective treatment, a harmful outcome, or even worse, death, for a patient or end user of the medicine.

In January 2010, the World Health Organization published an article on counterfeit drugs, describing the major health concern related with these illegal medicines. One counterfeit medicine for lowering blood sugar levels resulted in two deaths and nine hospitalizations in 2009 alone. In the United Republic of Tanzania in 2009, an antimalarial drug (Metakelfin), however lacking sufficient level of the active ingredient, was discovered in 40 pharmacies. This is particularly a problem in countries having weaknesses in their regulatory and enforcement systems. An estimated 1 in 4 packets of medicine sold in the street markets in developing countries is believed to be counterfeit. Over 50% of the medicines purchased over the Internet are from illegal sites that conceal their physical address, have been found to be counterfeit.

More generally, in this environment (and even that of more-developed countries), a pharmaceutical manufacturer typically has little to no interaction or review of the sales of their medicines. After distributing their products to representatives or directly to end users, the pharmaceutical company does not communicate with the user thereafter. Likewise, the medical distributor simply sells products, with no feedback from users or sales information pertaining to the users. There is no interaction between the users of the medicines and the providers (manufacturers or distributors) or the products. There is not an effective way of eliminating these counterfeit medicines, accordingly there is a need for a system that verifies the validity of medicines, for the sake of all parties involved, particularly the end user of the medicine.

Accordingly, there is a need for a system and method for accurately verifying the validity of medicines to ensure they are not counterfeit. The system and method should allow pharmaceutical companies and providers to observe when and where their products are selling. A desirable system would help reduce the availability of counterfeit medicines. There is a need for a system that obtains information pertaining to the users of the products to further analyze sales and distribution based on that information. It is desirable for such a system to perform comparative analysis of distribution and sales of their products. Further, communication between the pharmaceutical company and users is desirable to provide feedback and important messages from the company to users in addition to the user information to the companies. In such a system, the communication can be performed, for example, through cell phones, or other available portable or stationary electronic communication devices.

### SUMMARY OF THE INVENTION

There is provided a system and method for verifying the validity of medicines, as well as the tracking, monitoring, and otherwise managing medicines. The system and method performs related analysis of the data of product sales and distribution data. The system allows pharmaceutical companies to manage the sales and distribution of products based on a code associated with a particular batch of the medicine or the product itself. Each medicine includes an identifying product code used in managing the distribution and sales of the products, thereby identifying a particular product of a batch of products. These codes can be uploaded by users and pharmaceutical company representatives through any number of communication devices, including via a text message, a smartphone application, the Internet in general, or any other appropriate network. The code can be provided directly on an exterior surface of a bottle or other container for the medicine so it is readily available to a user.

The system includes a verification server that manages and controls the code data and information regarding sales of medicines. The verification server further analyzes the sales of medicines, verifying the validity of the medicines. The system further includes a management server that includes a plurality of applications running thereon for further managing the sales of medicines and related statistical data. The system allows pharmaceutical companies to send response and verification messages to users and representatives of the products. This improves feedback to users and pharmaceutical communication overall. The verification messages can be the same form as the codes that are uploaded by the users, or can be in a different form. For example, the verification messages can be in the form of text messages, electronic mail communications, voice mail messages, messages/interaction with a smartphone application, the Internet in general, or any other appropriate message provided through a network.

The system includes a verification database in which the codes that identify the medicines, and sales thereof, are stored. This database copies itself at set intervals on the verifications data only (without the codes) as a dashboard database, and the dashboard database is transmitted through the system network to a dashboard interface such that the updated statistics are available on the interface. The verification database is included within the verification system and the verification server communicates with the verification database to obtain any desired information. This decoupling between the codes and the verification data secures the system by transferring only verification data, and not codes, to the dashboard interface.

The dashboard interface is available on a display for managing the sales of medicines. The dashboard interface includes a plurality of application-specific buttons for viewing a specified analysis of the statistical sales data. The user can perform and review verifications of sales and comparisons of sales, or review response messages to be transmitted to users. The dashboard interface further provides graphical representations of the product sales, based on statistical data.

The method of the illustrative sales management system stores a code and associated data for medicines, to be used in managing the sales of the products. The method then tracks the sales of the products and initiates one of a plurality of applications to perform verifications, comparisons or review of responses generated to users.

The method can further perform statistical analysis on the sales of the medicines to report and display graphical representations, for example on the dashboard interface. The method also reports the sales of medicines to pharmaceutical companies, so that they can appropriately manage this information and report back to users via response messages.

### BRIEF DESCRIPTION OF THE DRAWINGS

The invention description below refers to the accompanying drawings, of which:

FIG. 1 is an overview block diagram of a system for managing the sales and distribution of medicines, according to an illustrative embodiment;

FIG. 2 is an overview block diagram detailing a dashboard interface implemented in the system for managing medicines, according to the illustrative embodiment;

FIG. 3 is a flow diagram showing an overall procedure for managing the sales and distribution of medicines, according to the illustrative embodiments;

FIG. 4 is a flow diagram detailing a procedure for the implementation of the dashboard user interface, according to the illustrative embodiments;

FIG. 5 is a flow diagram showing a procedure for the implementation of a response controller and manager for the system for managing medicines, according to the illustrative embodiments;

FIG. 6 is an exemplary browser screen display for the dashboard user interface, showing a home screen according to an illustrative embodiments;

FIG. 7 is an exemplary browser screen display for the dashboard user interface, showing a verifications graph by total sales, according to the illustrative embodiments;

FIG. 8 is an exemplary browser screen display for the dashboard user interface, showing a verifications graph by region, according to the illustrative embodiments;

FIG. 9 is an exemplary browser screen display for the dashboard user interface, detailing the products tab option of the display, according to the illustrative embodiments;

FIG. 10 is an exemplary browser screen display for the dashboard user interface, detailing the batches tab option of the display, according to the illustrative embodiments;

FIG. 11 is an exemplary browser screen display for the dashboard user interface, detailing the dates tab option of the display, according to the illustrative embodiments;

FIG. 12 is an exemplary browser screen display for the dashboard user interface, showing a cross-selling graphical display, according to the illustrative embodiments;

FIG. 13 is an exemplary browser screen display for the dashboard user interface, showing responses by region, according to the illustrative embodiments; and

FIG. 14 is an exemplary browser screen display for the dashboard user interface, showing responses by product, according to the illustrative embodiments.

### DETAILED DESCRIPTION

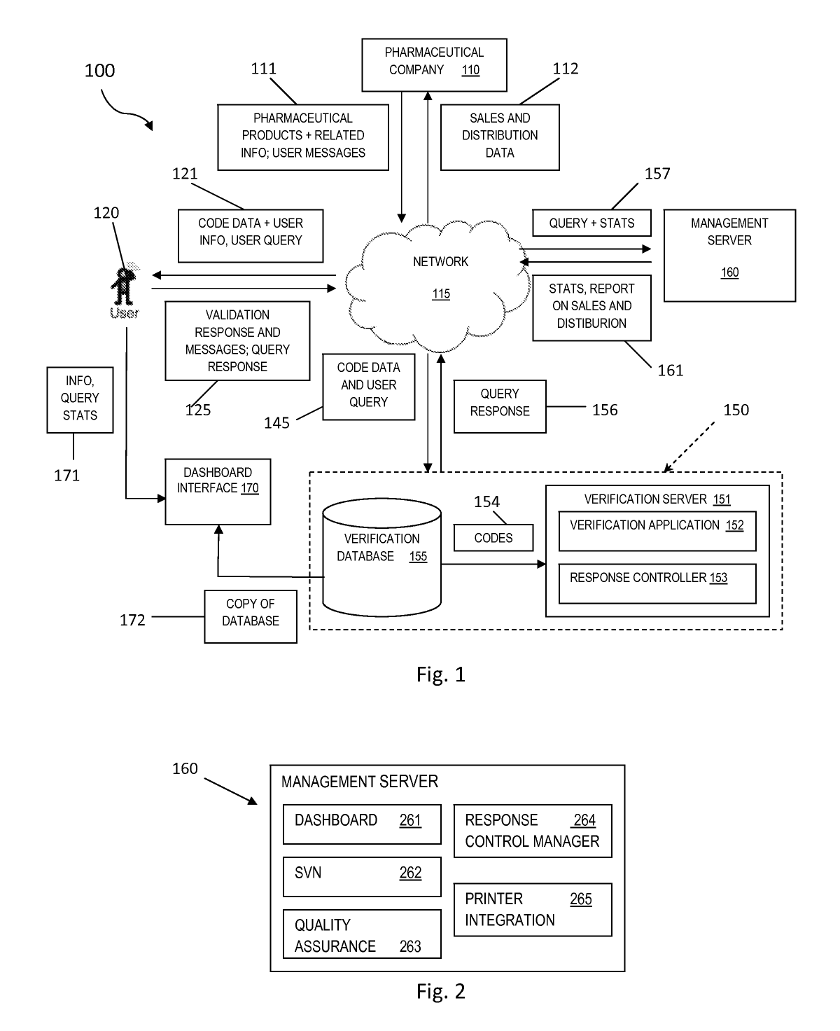

There is provided a system and method for verifying the validity of medicines, as well as tracking, monitoring, and otherwise managing medicines, and related analysis of the sales and distribution data. As shown in FIG. 1, a medicine management system 100 is provided in which a pharmaceutical company 110 provides medicines to users and consumers. “Users” as defined herein represent either end users of the medicines, or representatives for the pharmaceutical company that assist in the distribution and sales of the medicines to end users.

As shown in FIG. 1, a pharmaceutical company 110 transmits the products and related information (including codes identifying the products) via datastream 111 to a network 115. The verification codes which identify a particular product or a batch of medicine is provided by the pharmaceutical company. Each medicine or product includes an identifying verification product code used in managing distribution and sales, and is also used to identify a particular product. The network 115 can comprise any appropriate communication network including a local area network (LAN), the broad worldwide Internet, or cellular telephones used through a cellular tower network. As described in greater detail hereinbelow, the system provides the pharmaceutical company with feedback based on the distribution and, more importantly, sales of the medicines via datastream 112. This information allows the pharmaceutical company to develop user messages based on this information, which is transmitted to the network via datastream 111, to be sent to a particular user or consumer. The code data uploaded to the system, as well as the response “verification” messages transmitted to the user, can be in any appropriate form of communication through any network. For example, the messages can be standard text message (SMS (short messaging service), MMS (multimedia messaging service) or other), smartphone application messages, or through the Internet in general through an electronic internet-based message.

A user 120 of the system 100 transmits code data about the medicine, as well as user information, containing particular information about the user via datastream 121. The user can be an end user or consumer, seeking to verify the validity and safety of their medications. A user transmits the code data to verify the authenticity of the medicines, based upon feedback from the various components of the system 100.

According to the system 100, the user submits a query via datastream 121 to the network 115 to perform a call in function to verify the validity, or one of a plurality of other applications pertaining to the system. The code data and user query are transmitted via datastream 145 to a verification system 150. The verification system includes a verification server 151 that has a verification application 152 and response controller 153 running thereon. The verification server 151 receives the query responses and codes to perform appropriate control and verification of data to verify the validity of the medicines. The verification server 151 also stores the codes 154 into the secure verification database 155, within the verification system 150. The statistics and reports on sales and distribution of the medicines generated by the verification system are transmitted back to the network 115 via datastream 156. These are then transmitted back to a user 120 via datstream 125 and a pharmaceutical company 110 via datastream 112. While the various servers and/or applications are depicted at specific locations and perform specific tasks, the relative positions and division of tasks between the various entities is highly variable.

User queries and statistics are transmitted via datastream 157 to a management server 160. The management server 160 includes a plurality of applications that perform a multitude of functions, described in greater detail with reference to FIG. 2. The management server 160 performs one of a multitude of functions, and this information, as statistics, reports on sales and distribution, or other pertinent data, is transmitted via datastream 161 to the network 115, to be further transmitted to pharmaceutical companies 110 and users 120.

On a periodic basis, the secure verification database 155 selectively copies itself (no codes are transferred, only the verification data) to provide a copy to the users, for example, as a dashboard database at a dashboard interface. The decoupling between codes and verification data secures the system by transferring only verification data, and not codes, to the dashboard interface. A dashboard database (the copy of the database without codes) is transmitted to the dashboard user at a dashboard interface 170 via datastream 172. These updated statistics therefore reside on the management server 160 through the dashboard user interface (see FIG. 2). A query made by the user, via datastream 171, at the dashboard user interface 170 reflects the updated statistics data.

The verification server and the management server can comprise any appropriate server or computing architecture, including a computer program running as an application or other service, a physical computer dedicated to running applications, and/or a software/hardware system of components, as described herein and otherwise readily apparent to those of ordinary skill.

Reference is now made to FIG. 2, showing a detailed schematic view of the management server 160 of FIG. 1. The management server 160 includes a plurality of applications, functions and/or additional servers for performing the various tasks of the system for managing sales of medicines. These applications include, for example, a dashboard application 261, SVN (Version Control) application 262, Quality Assurance application 263, Response Control Manager application 264, and Printer Integration application 265. It is expressly contemplated that while these are shown and described as applications residing on the management server, each can be represented as a separate server or as a virtual machine, or an application running on a server or virtual machine. Likewise, the distribution of tasks between the various applications and/or servers is highly variable. Furthermore, the placement and location of servers and applications is highly variable.

The dashboard 261 is an analytics tool used to provide the total verifications, daily verifications, regional verifications, product verifications and cross-sell verifications, among others, for the system for verifying medicines. The SVN application 262 is a version-control system that is used to maintain current and historical versions of files. Can be used for quality assurance and other production environments. The quality assurance application 263 analyzes data to ensure quality assurance of the overall system. The response control manager 264 coordinates with the verification server 150 to manage responses based on products and/or batches of products. The printer integrator 265 can be a server or application that connects with the pharmaceutical company to transfer codes online. The printer integrator 265 also coordinates with the verification system 150 to verify the codes.

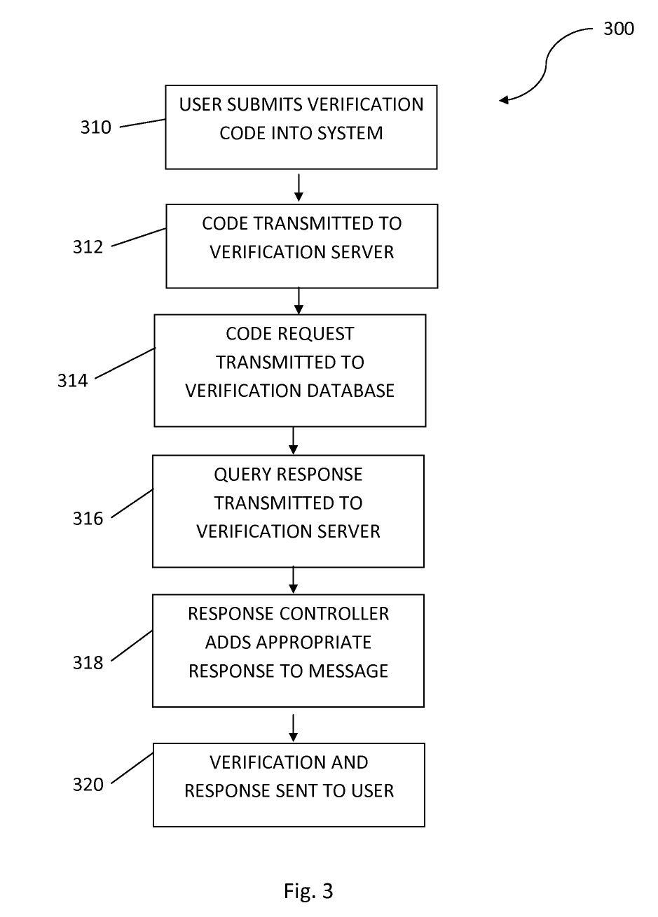

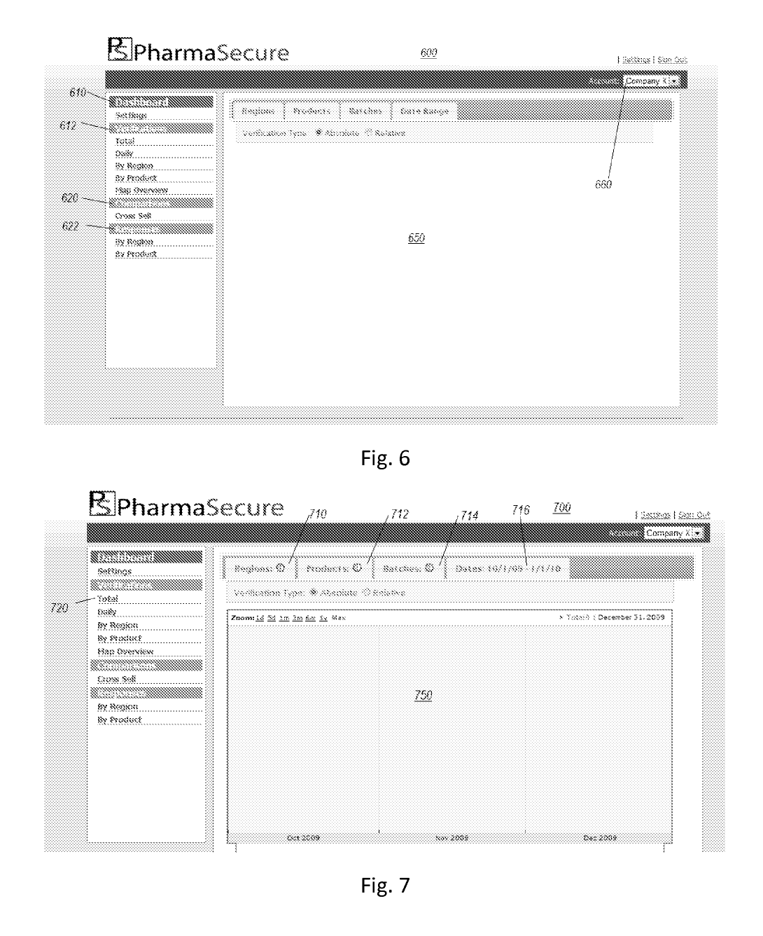

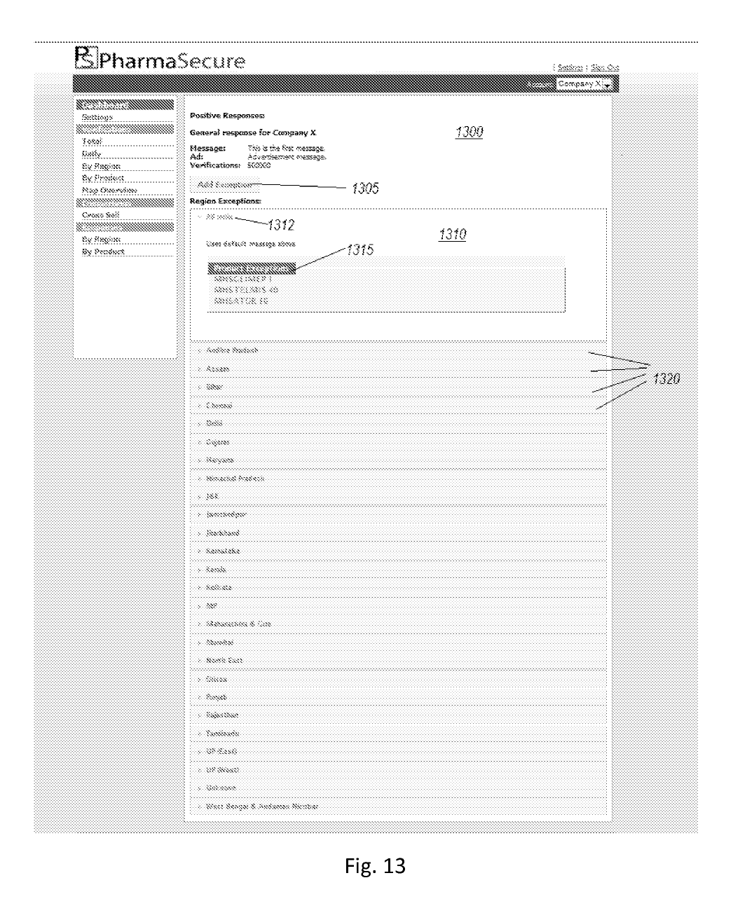

The statistics and reports on the sales and distribution of the medicines are generated by the verification system according to the procedures and methods disclosed herein and discussed in greater detail with reference to the flow charts of FIGS. 3-5 and exemplary browser screen displays of FIGS. 6-14 for the dashboard user interface. The dashboard user interface of FIGS. 6-14 allows a user and/or pharmaceutical company to effectively manage and monitor the generated data based upon distribution and sales of the medicines.

Referring now to FIG. 3, a procedure 300 is shown for the user interaction with the system for verification and managing medicine sales. The user verification procedure 300 begins at step 310 when the user submits a verification code into the system. This verification code is representative of an individual medicine and/or a batch (group) of medicines. This code is then transmitted to the verification server, as well as information about the user, at step 312, to verify the validity of the medicines. The procedure then transmits a code request to the verification database at step 314 to obtain information about the medicine. This information is used in tracking the sales of the medications. The query response is then transmitted back to the verification server at step 316.

Based upon the query response, the verification server prepares a message for the user and the response controller application (running on verification server) adds an appropriate response to the message at step 318. The verification and response are then sent to the user via the network at step 320. These are used to communication a verification (or lack thereof) to a user. The verification messages can be presented to the user through any appropriate system described herein, including text messages through a cellular telephone network, smartphone application messages, and internet-based messages, among others.

There can be three types of responses generated for a user. The first is a valid response for up to “n” notifications, where “n” is any number of medicines. An example of this first response is: “MCN labs authenticates genuine product of [Product Name], batch [Batch Number]. Thank you for your patronage. Service provided by PharmaSecure.” The second is an “invalid” response, which is returned if the customer enters an invalid code. An example of this second response is: “The code typed ‘---’ is incorrect. Please retype the code or contact chemist for further assistance. Service provided by PharmaSecure.” The third is an “oververified” code, in which the number of verifications is excessive for that particular code, thereby indicating it is not a valid medicine. An example of this third response: “Warning! Expired code! Please contact the chemist for further assistance. Service provided by Pharmasecure.”

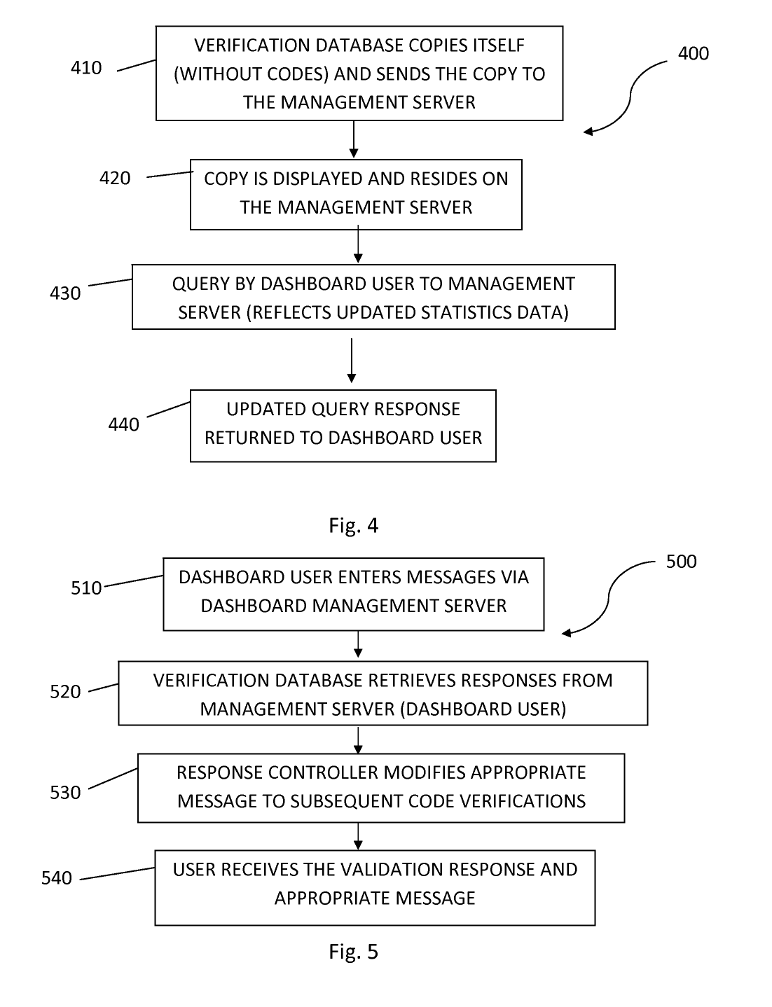

FIG. 4 shows a dashboard user procedure 400 for interaction with the management server according to an illustrative embodiment. The verification database copies itself and sends a copy to the management server at step 410. The copy is displayed and resides on the management server at step 420. This allows the updated statistics to reside on the management server. A query for updated data is sent to the management server at step 430 to reflect the updated statistics data on the dashboard user. The dashboard user requests updated data from the management server at step 430. The updated query response is then returned to the dashboard user at step 440. Therefore, updated data is present to the dashboard user.

FIG. 5 details a message management procedure 500 according to the illustrative system for managing medicine sales. The procedure 500 begins at step 510 when a dashboard user enters messages via the dashboard application on the management server, for example a request for verification of the validity and safety of a medicine. The messages include verification, error or expired response messages. The verification database then retrieves responses from the management server at step 520. These are pulled from the management server by the verification database on a periodic basis.

Then at procedure step 530 the response controller of the management server modifies an appropriate message for the dashboard user to include subsequent code verifications. The management server includes applications for performing various statistical analysis and review of the sales of the medicines. These are included in the response to the user to provide a message with details on the particular statistical data of the sales of the medicines. The user then receives the validation response and appropriate message at step 540, thereby validating (or finding a lack of validation) for the medicine.

Reference is now made to FIGS. 6-14, detailing exemplary browser screen displays for the dashboard user interface of the illustrative system. Each of the exemplary browser screen displays can be on an appropriate display of any device, including a browser screen display of a website, to be displayed on a computer, cellular telephone, PDA, or other portable handheld electronic device. Other devices employed by the systems and networks described herein should be apparent to those having ordinary skill and are readily applicable thereto. The browser screen displays can be for use by the users or consumers of medicines to receive messages and important data, as well as by the pharmaceutical companies and representatives of the pharmaceutical companies.

FIG. 6 shows a home page screen 600, or welcome screen, according to an illustrative embodiment for an exemplary browser screen display. The home screen 600 provides users with a plurality of applications for managing and reviewing the sales of medicines, including the dashboard settings 610, verifications 612, comparisons 620 and responses 622. Based upon the particular application that is selected, the graphical display 650 contains a particular graphical representation of the selected medicines. A user is provided with a drop-down box 660 that allows them to select the particular company or representative for which the data is displayed. This allows multiple companies or representatives to be managed simultaneously in one convenient dashboard interface.

A user selecting the dashboard link 610 is directed to a page for determining the settings of the dashboard interface system. The verifications option 612 allows a user to view the total verifications (720 of FIG. 7), daily verifications, and verifications by region, verifications by product and a map overview. These provide various graphical representations in the region 650 of the screen.

As shown in FIG. 7, a verifications graph by total verifications is shown, by selecting the Total link 720. As shown in the browser screen 700, the user is provided with a plurality of tabs for selecting the particular group to be analyzed. There is provided a regions tab 710 to select the regions included in the analysis, a products tab 712 to select the products included in the analysis, a batches tab 714 to select the various batches included, and a dates tab 716 to select the dates for which analysis occurs. The results of the verifications, based on the selected parameters, are displayed in the graphical representation 750.

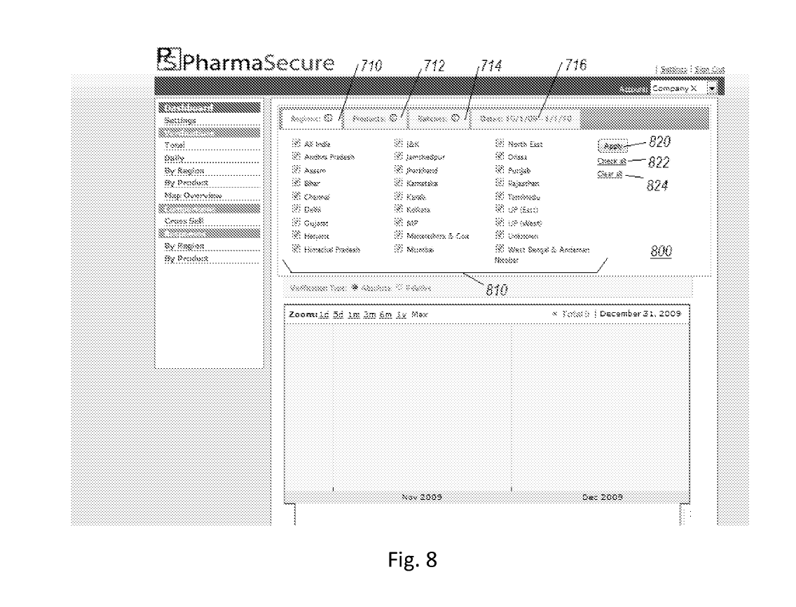

Selecting the regions tab 710 causes a drop down screen 800 to be displayed, as shown in FIG. 8. This provides a user with a plurality of check boxes 810 for each of the regions available for analysis and sales. A user can select the apply button 820 to apply the particular regions of interest to the graphical representation 750. A user can also select a check all link 822 to select all of the regions available, or select a clear all link 824 to deselect particular regions.

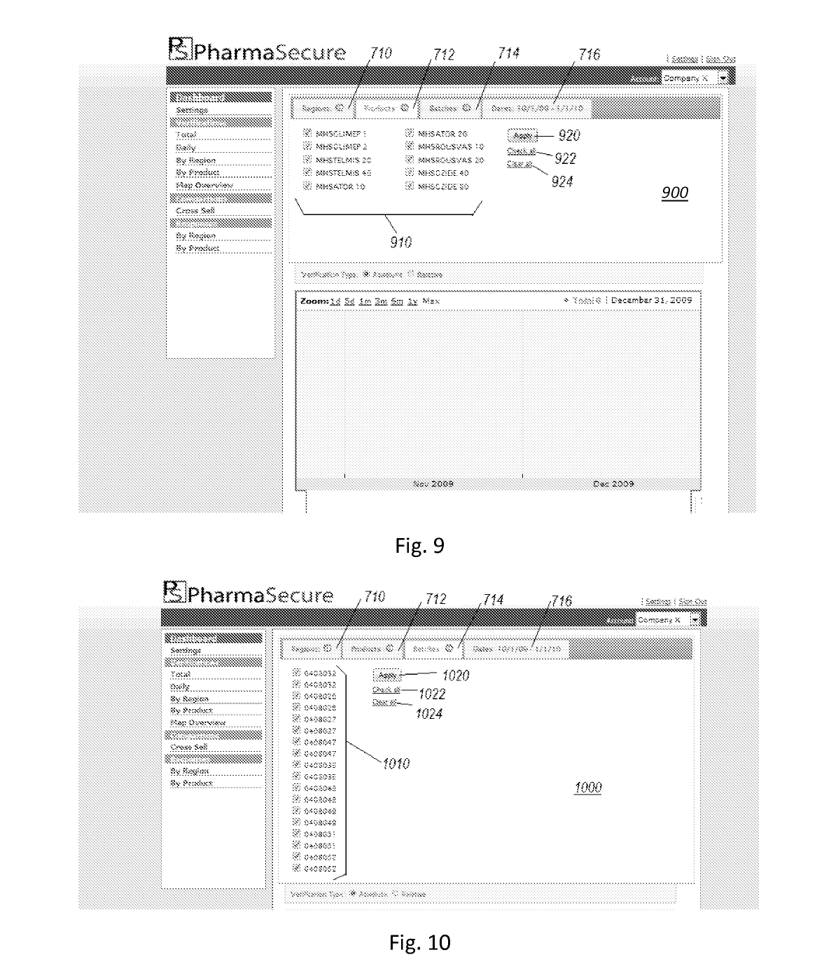

As shown in FIG. 9, selecting the products tab 712 causes a drop down screen region 900 to be displayed. This provides a dashboard user with a plurality of check boxes 910 for each of the products available for analysis and to manage the sales of the selected medicines (selected by checking the boxes 910). A user can select the apply button 920 to apply the particular medicines of interest to the graphical representation 750. A user can also select a check all link 922 to select all of the products for analysis, or select a clear all link 924 to deselect particular products.

By selecting the batches tab 714, a drop down screen 1000 is displayed. This provides a list of check boxes 1010 to select the various batches of medicines to be analyzed. A user can select the apply button 1020 to apply the particular medicine batches of interest to the graphical representation 750. A user can also select a check all link 1022 to select all of the batches for analysis, or select a clear all link 1024 to deselect particular batches for the medicines.

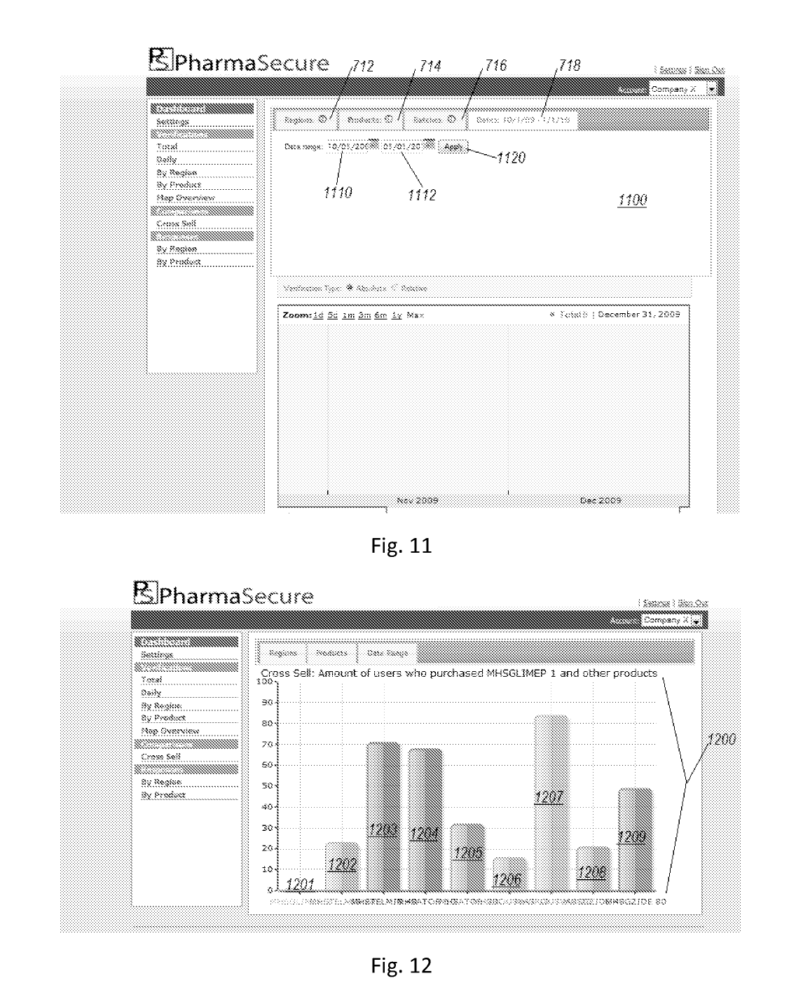

A user selecting the dates button 716 causes a drop down screen 1100 to be displayed, as shown in FIG. 11. This allows a user to select the particular date range for which verifications of sales of medicines are performed. A user can enter a beginning date in box 1110 and an end date in box 1112. The user can then select the apply box 1120 to select the date range for which analysis is performed.

A dashboard user can also perform comparisons by selecting the link 620 of FIG. 6, which directs a user to the browser screen display shown in FIG. 12. This allows users to compare sales of a particular product as compared with a plurality of other products. A user can accordingly view the cross-sales of a particular product. For example, as shown in FIG. 12, the various product sales are represented by the graphical region 1200, which includes databars 1201, 1202, 1203, 1204, 1205, 1206, 1207, 1208, and 1209. These bars each show the sales of a particular product as compared with sales of other products to show cross sales.

Users can also set the responses that will be transmitted from the pharmaceutical company, either by region or by product. Selecting responses by region directs a user to the screen display 1300 that provides messages for a particular company. As shown. A user can add further exceptions to the messages by selecting the add exception box 1305. A user can select a particular region exception by selecting the name, for example for all India link 1312. This presents a user with the screen box 1310 in which a plurality of product exceptions 1315 are listed as exceptions to the response messages.

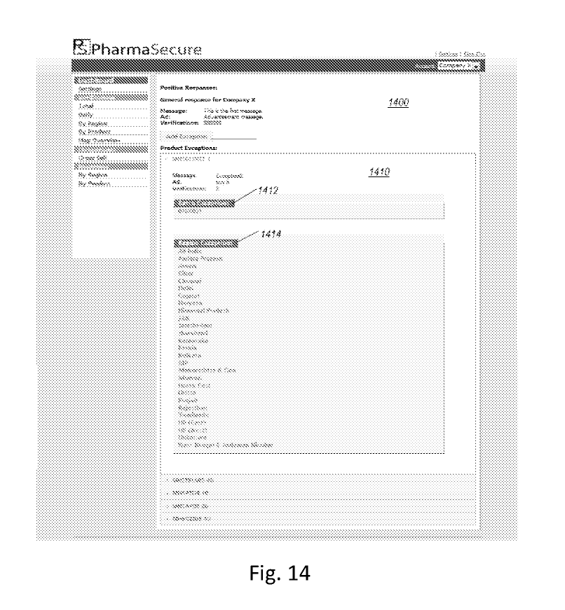

The responses can also be reviewed by product, for example as shown in FIG. 14. The responses by region screen 1400 includes an add exception box 1405 that allows users an opportunity to add more exceptions to the response messages. A user can select a particular product, for example MHSGLIMEP1, which opens drop down screen 1410. This lists a plurality of batch exceptions 1412 and region exceptions 1414 for the response messages.

These and other applications and advantages should be clear to an ordinarily skilled person. The systems and methods herein improve review of sales and medicines and the communication between a pharmaceutical company and users of the medicines.

The foregoing has been a detailed description of illustrative embodiments of the invention. Various modifications and additions can be made without departing from the spirit and scope of this invention. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. For example, the procedures have been described with reference to managing medicines, particular drugs provided by a pharmaceutical company. However, the teachings herein are readily applicable to any medicine that is sold or distributed, for which data is available. Accordingly, this description is meant to be taken only by way of example, and not to otherwise limit the scope of this invention.

What is claimed is:

1. A system for validating sales of medicines comprising:
a verification server that receives code data regarding the sales of at least one medicine, transmitted over a network by a user seeking validity of the at least one medicine, and wherein the verification server transmits a verification message to the user;
a management server having an application running thereon that analyzes the validity of the at least one medicine based upon the code data transmitted by the user; and
a verification database in which codes corresponding to the sales of medicines are stored.

2. The system as set forth in claim 1 further comprising:
a dashboard interface display that provides a pharmaceutical company with feedback of the sales of medicines that they have distributed.

3. The system as set forth in claim 1 wherein the at least one medicine is stored in a container that includes the code data directly on an exterior surface thereof.

4. The system as set forth in claim 1 wherein the network comprises the internet.

5. The system as set forth in claim 1 wherein the network comprises a cellular telephone network.

6. The system as set forth in claim 1 wherein the verification message is transmitted to a user as one of a text message, a smartphone application and an internet-based message.

7. A method for validating sales of medicines comprising the steps of:
obtaining code data, transmitted to a verification server over a network, to validate at least one medicine; and
analyzing, by the verification server, the at least one medicine based upon information provided by the pharmaceutical provider of the at least one medicine.

8. The method as set forth in claim 7 further comprising the step of transmitting a verification message when the at least one medicine is validated by the verification server.

9. The method as set forth in claim 7 further comprising the step of submitting a request to a management server to perform a comparison of the sales of at least one of the plurality of medicines to at least another one of the plurality of medicines.

10. The method as set forth in claim 7 further comprising the step of performing statistical analysis on the sales of the plurality of medicines to obtain a comparison of the sales of the medicines.

11. The method as set forth in claim 7 further comprising the step of storing the code data into a verification database to provide a secondary source for the verification.

12. The method as set forth in claim 7 wherein the network comprises the internet.

13. The method as set forth in claim 7 wherein the network comprise a cellular telephone network and the message are transmitted as one of a text message, a smartphone application and an internet-based message.

14. The method as set forth in claim 7 wherein the code data is provided to the user as displayed on a container of the at least one medicine.

15. A graphical user interface display comprising:
a plurality of application-specific buttons for viewing a specified analysis of statistical data corresponding to sales of medicines;
a plurality of selector tabs for selecting particular parameters for which the analysis of statistical data occurs to view an effect of the particular parameters on the analysis of statistical data; and
a graphical display that provides of the analysis of statistical data performed on the sales of medicines, thereby providing feedback to one of a user of the medicines and a pharmaceutical company of the medicines.

16. The graphical user interface display of claim 15 wherein the particular parameters for which the statistical analysis will occur include one of a regional area, a product control and a date range.

17. A medicine container provided to a user, the medicine container comprising:
a verification code provided by a pharmaceutical company used in managing distribution and sales of a particular product to identify the particular product, the verification code adapted to be stored in a verification database for verification of distribution and sales of the particular product; and
wherein the verification code is adapted to be submitted by a user to a verification server to validate the particular product based upon the verification code provided to the verification server as compared to the verification code stored in the verification database.

18. The medicine container of claim 17 wherein the verification code is adapted to be transmitted to the verification server as one of a text message, a smartphone application message and an internet-based message.
